(19) United States
(12) Patent Application Publication
(10) Pub. No.: US 2023/0131906 A1
(43) Pub. Date: Apr. 27, 2023

(54) BACTERIUM OF THE CHRISTENSENELLACEAE FAMILY AND COMPOSITION CONTAINING SAME FOR THE PREVENTION AND/OR TREATMENT OF PATHOLOGICAL MUSCLE LOSS OR OF A DISEASE CHARACTERISED BY PATHOLOGICAL MUSCLE LOSS

(71) Applicant: YSOPIA BIOSCIENCES, BORDEAUX CEDEX (FR)

(72) Inventors: Georges RAWADI, PARIS (FR); Sandrine CLAUS, BORDEAUX (FR); Laure RINALDI, BORDEAUX (FR)

(21) Appl. No.: 17/915,600

(22) PCT Filed: Jun. 25, 2020
(86) PCT No.: PCT/EP2020/067892

§ 371 Date: Oct. 6, 2022

(30) Foreign Application Priority Data
Jun. 25, 2019 (FR) FR1906843

(51) Int. Cl. A61K 35/741; A61P 21/06; A61K 45/06

(52) U.S. Cl. CPC A61K 35/741; A61P 21/06; A61K 45/06; A61K 2035/115

(57) ABSTRACT

The invention relates to bacteria of the Christensenellaceae family and to compositions including same for use in the prevention and/or treatment of pathological muscle loss and/or of at least one disease characterized by muscle loss in humans or animals.

### TECHNICAL FIELD

The invention relates to muscle preservation. In particular, the invention relates to bacteria specific to the gut microbiota and to compositions containing them for preventing and/or treating pathological muscle loss or a disease characterized by pathological muscle loss.

### PRIOR ART

Pathological muscle loss is well known to those skilled in the art and refers to muscular atrophy, a decrease in muscle volume (Gordon et al. Therapeutic approaches for muscle wasting disorders. Pharmacol Ther. 2007 March; 113(3):461-87). This muscle loss generally concerns the striated skeletal muscles, which are the muscles under voluntary control. Several pathologies are characterized by muscle loss, in particular amyotrophy, pre-sarcopenia, sarcopenia, rhabdomyolysis, cachexia.

Amyotrophy is a decrease in the volume of skeletal and cardiac striated muscles, that is to say, the number of fibers composing these muscles (long cells) decreases.

Sarcopenia is a syndrome characterized by age-related muscle loss that can lead to deterioration in muscle strength and physical performance.

Rhabdomyolysis is a disease in which cells of the skeletal muscles rapidly degrade and release their contents into the bloodstream. Some of these breakdown products of damaged muscle cells are harmful to the kidneys and can lead to kidney failure in particular. The disease is characterized by several symptoms, such as muscle aches, vomiting and confusion, and can be life threatening in some cases. Some animals may be affected, in particular horses.

Cachexia is a profound weakening of the body characterized by weight loss, fatigue and muscular atrophy in particular, linked to very significant malnutrition.

Muscular atrophy can be caused in particular by the synthesis of norleucine in the body. Other markers of muscle wasting are known as albumin, pre-albumin, C-reactive protein and myoglobin.

Norleucine is a non-proteinogenic amino acid that, when present, takes the place of another amino acid, methionine, during protein synthesis. This replacement of methionine by norleucine is toxic for the cells, and it in particular prevents the renewal of muscle cells, decreases the growth of muscle fibers and reduces muscle development (Bogosian et al. Biosynthesis and Incorporation Into Protein of Norleucine by Escherichia Coli, J Biol Chem 1989 Jan. 5; 264(1):531-9. Norleucine particularly affects organs where protein replacement is important, such as muscle. Thus, the muscles experience low-dose norleucine toxicity. As an example, Jeffery Escobar et al. 2010 demonstrated that the administration of leucine to piglets has a positive effect on protein synthesis in skeletal muscle and that norleucine does not have a positive effect on protein synthesis in muscle. They also show that the chemical structure of leucine gives it these properties.

Norleucine can be synthesized by bacteria in the side product pathway of the branched chain amino acid synthesis pathway from pyruvate and alpha-ketobutyrate.

Currently, the toxicity of norleucine is studied very little and no treatment for pathological muscle loss or a disease characterized by pathological muscle loss is based on a reduction in norleucine toxicity, despite the involvement of this amino acid in ubiquitous metabolic pathways.

Albumin is a soluble protein that is measured to check the amount of protein in the blood. A low albumin concentration is a marker of malnutrition, which is accompanied by significant muscle loss.

C-reactive protein is a protein that shows the patient's inflammatory state, which is observed during muscle loss due to cachexia in particular. A high concentration of C-reactive protein is a marker of cachexia, which is accompanied by significant muscle loss.

Myoglobin is a protein capable of transporting oxygen to the muscles, therefore essential for the work and development of the muscles. A high concentration of plasma myoglobin is a marker of muscle damage or rhabdomyolysis in polytrauma, infectious diseases, muscular dystrophy, myopathy.

Furthermore, existing treatments for pathological muscle loss or diseases characterized by pathological muscle loss are not effective. Indeed, these essentially consist of physical exercises during repeated sessions several times a week, an optimal protein intake to stimulate protein synthesis, the installation of an infusion containing a saline solution, alkalinization of the urine, dialysis, surgery of the injured region, which does not provide a satisfactory result. In addition, the treatments are often heavy, like for the treatment of rhabdomyolysis, which consists of abundant intravenous infusions, and which may require dialysis or hemofiltration in more serious cases.

One article focused on the study of the gut microbiota in patients with primary sarcopenia, and in particular the effects of physical activity on the microbiota. The effects of administering probiotics such as FOS and inulin to improve symptoms of muscle weakness and sarcopenia have also been discussed, although there is no evidence of a distinct gut microbiota in patients with sarcopenia (Andrea Ticinesi et al. Aging gut microbiota at the cross-road between nutrition, physical frailty, and sarcopenia: Is there a gut-muscle axis?. 2017). However, the study does not show any particular interest for certain strains, nor any use of bacteria according to the invention.

Thus, there is a significant need for an effective treatment of pathological muscle loss and diseases characterized by pathological muscle loss that is capable of acting on the various markers at the origin of the disease, and in particular on the synthesis of norleucine, that is easy to administer and that has no side effects.

### SUMMARY OF THE INVENTION

This is the objective of the present invention, which, to respond to this need, focuses on the use of particular bacteria of the human gut microbiota, namely bacteria of the family Christensenellaceae.

Bacteria of the family Christensenellaceae, including the genus Christensenella, have already been studied and described. This is the case in particular for Christensenella minuta, Christensenella massiliensis and Christensenella timonensis. Christensenella minuta in particular was described for the first time in 2012. In 2014, a study showed that it was the most heritable taxon in a cohort of British twins and that its presence is associated with a low body mass index. This correlation between Christensenella minuta and low body mass index was then observed in a dozen studies published since 2014 in geographically diverse populations. Finally, patent application WO/2018/162738 describes the use of bacteria from the Christensenellaceae family in the treatment of obesity and excess weight

Surprisingly, according to the invention, the bacteria of the Christensenellaceae family, when they are administered to humans or animals, are capable of acting on the markers of pathological muscle loss to prevent or treat this disease or diseases characterized by muscle loss, such as amyotrophy, sarcopenia, pre-sarcopenia, rhabdomyolysis, cachexia.

Therefore, the subject of the invention is a bacterium of the family Christensenellaceae, for its use in the prevention and/or treatment of muscular atrophy in humans or animals.

Advantageously, such a bacterium, when it is administered to a human being or an animal exhibiting pathological muscle loss or a disease characterized by muscle loss, is capable of acting on the molecules produced in excess during this pathological state such as in particular norleucine, and also possibly against albumin and/or pre-albumin and/or protein C and/or myoglobin, allowing an increase in protein synthesis and production of muscle cells to regain lost muscle mass.

In addition, it is a bacterium naturally present in the gut microbiota and whose administration does not cause side effects.

Preferably, the bacteria for use according to the invention are administered within compositions. Thus, the invention also relates to compositions comprising at least one bacterium of the family Christensenellaceae for its use in the prevention and/or treatment of pathological muscle loss and/or a disease characterized by pathological muscle loss in humans or animals.

Other features and advantages will become apparent from the detailed description of the invention that follows.

### DETAILED DESCRIPTION OF THE INVENTION

### Definitions

Within the meaning of the invention, “pathological loss of muscle mass” or “muscle loss” or “pathological muscle loss” means an abnormal, pathological decrease in muscle mass due to a decrease in protein synthesis, in particular of skeletal muscle mass.

Within the meaning of the invention, “disease characterized by pathological muscle loss” or “disease characterized by muscle loss” means a disease whereof one of the symptoms is a loss of muscle mass. These may in particular include amyotrophy, sarcopenia, pre-sarcopenia, rhabdomyolysis or cachexia.

Within the meaning of the invention, “marker” of a disease means a molecule or a substance whose assay makes it possible to follow the evolution of said disease.

### Bacteria According to the Invention

The invention relates to the use of at least one bacterium of the Christensenellaceae family in the prevention and/or treatment of pathological muscle loss and/or a disease characterized by pathological muscle loss in humans or animals.

The invention therefore relates to a bacterium of the Christensenellaceae family for its use in the prevention and/or treatment in humans or animals of pathological muscle loss or of a disease characterized by muscle loss chosen in particular from amyotrophy, sarcopenia, pre-sarcopenia, rhabdomyolysis and cachexia, in particular in humans or animals exhibiting pathological muscle loss or a disease characterized by pathological muscle loss with a synthesis of at least norleucine and/or of at least one marker chosen from: albumin and/or pre-albumin and/or C-reactive protein and/or myoglobin.

According to the invention, the bacteria of the Christensenellaceae family, when they are administered to a human or an animal exhibiting a muscle loss or a disease characterized by muscle loss, are capable of acting on the molecules produced in excess during this pathological state, in particular on the production of norleucine and/or of at least one marker chosen from: albumin and/or pre-albumin and/or C-reactive protein and/or myoglobin.

In the case of pathological muscle loss, in particular in diseases characterized by muscle loss that exhibit synthesis of norleucine, and/or synthesis of at least one marker chosen from: albumin and/or pre-albumin and/or C-reactive protein and/or myoglobin, their decrease is a sign of increased protein synthesis and increased muscle cell production.

When pathological muscle loss or disease characterized by pathological muscle loss presents with hyperproduction of norleucine, its decrease is a sign of reduced pathways for the synthesis of branched chain amino acid byproducts, that is to say, this production is less stimulated. From then on, the production of excessive norleucine responsible for its toxicity is slowed down and the system gradually returns to normal. The physiological mechanism of muscle synthesis normalizes and muscle development increases.

The useful bacteria according to the invention are administered to humans or animals in an amount effective for an action on at least one of these markers of pathological muscle loss, i.e., to reduce the production of at least one of these markers in the body.

According to a suitable embodiment, the bacterium or bacteria are administered at a dose of 10⁹ to 10¹² colony-forming units (CFU) per day, regardless of the weight of the person or animal. It will preferably be a single dose, i.e., administered once daily, or a dose before each meal (three times a day).

The useful bacterium or bacteria according to the invention are bacteria of the Christensenellaceae family, preferably of the genus Christensenella. It may be, in particular, Christensenella massiliensis, Christensenella timonensis and/or Christensenella minuta. According to a particularly suitable variant, it is Christensenella minuta.

These bacteria can be isolated from human stools for example according to the protocols published by Morotomi et al. 2012 (Morotomi, M., Nagai, F. & Watanabe, Y. Description of Christensenella minuta gen. nov., sp. nov., isolated from human faeces, which forms a distinct branch in the order Clostridiales, and proposal of Christensenellaceae fam. nov. INTERNATIONAL JOURNAL OF SYSTEMATIC AND EVOLUTIONARY MICROBIOLOGY 62, 144-149 (2012)) and NDongo et al. 2016 (Ndongo, S., Dubourg, G., Khelaifia, S., Fournier, P. E. & Raoult, D. Christensenella timonensis, a new bacterial species isolated from the human gut. New Microbes and New Infections 13, 32-33 (2016)). These documents also describe the culture methods of the bacteria that are useful according to the invention.

### Compositions

The useful bacteria according to the invention are preferably administered to the human or animal in a composition.

Thus, the invention also relates to compositions comprising at least one bacterium of the family Christensenellaceae for its use in the prevention and/or treatment of pathological muscle loss and/or a disease characterized by pathological muscle loss in humans or animals, in particular in persons or animals exhibiting norleucine synthesis and/or at least one marker chosen from: albumin and/or pre-albumin and/or C-reactive protein and/or myoglobin.

The bacteria are present in an effective amount in the composition, allowing an effect on the loss of muscle mass of the treated persons or animals.

Preferably, the useful composition according to the invention comprises 10⁶ to 10¹² colony-forming units (CFU) of bacteria of the family Christensenellaceae per daily dose of composition to be administered. Preferably, this corresponds to a daily dose of bacteria to be administered, regardless of the weight of the person or the animal. Preferably, this daily dose is administered once per day.

The useful composition according to the invention may be in liquid form. It may in particular comprise bacteria of the Christensenellaceae family and a culture medium for said bacteria that makes it possible to preserve them, such as, for example, Columbia anaerobic medium enriched with sheep blood, or an equivalent medium not containing an animal byproduct.

When the compositions are in liquid form, they are preferably frozen, maintained at −20° C. in a sealed bag.

According to one variant, the useful composition according to the invention may be in solid form. In this case, the bacteria may be present in freeze-dried form, and the compositions may also comprise excipients such as, for example, microcrystalline cellulose, lactose, sucrose, fructose, levulose, starches, stachyose, raffinose, amylum, calcium lactate, magnesium sulphate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, sorbitol and/or glycerol.

The useful compositions according to the invention may in particular be in the form of powder, microencapsulated powder, gelcap, capsule, tablet, lozenge, granules, emulsion, suspension or suppository. According to a particularly suitable embodiment, they may be in a gastro-resistant form, such as a coated tablet containing microencapsulated bacteria.

When the compositions are in solid form, they are preferably packaged in capsules or in a coating hermetically sealed against light and oxygen, maintained at an ambient temperature of between 15° C. and 40° C. and a humidity level between 3% and 70%.

The bacteria can be used alive, or inactivated, for example by heat, exposure to an appropriate pH, gamma radiation or high pressure.

They can all be alive or all inactivated.

Preferably, at least one part is made up of living bacteria are alive, in particular at least 50% (by number), even more preferably at least 90% (by number).

Thus, according to a suitable embodiment, the bacteria present in the useful composition according to the invention are at least 50% living bacteria (by number), preferably at least 90% living bacteria (by number), and even more preferably all living.

The useful bacteria according to the invention, and in particular the compositions that include them, can be administered orally, topically, through the lungs (inhalation) or rectally.

The useful compositions according to the invention, in addition to the useful bacteria according to the invention, can comprise other compounds, such as:
at least one probiotic, and/or
at least one bacterium producing lactic acid, which makes it possible to create an anaerobic environment favorable to Christensenellaceae, such as at least one bacterium chosen from bacteria of the genus Lactobacillus spp., Bifidobacterium spp., Streptococcus spp. and/or at least one other organism promoting the anaerobic conditions necessary for the survival of Christensenellaceae, such as at least one yeast chosen from Saccharomyces spp. or microorganisms of the Methanobacteriaceae family, and/or
at least one bacterium associated with the Christensenellaceae ecosystem, since they facilitate their survival in the intestine, such as at least one bacterium chosen from bacteria of the phylum Firmicutes, Bacteroidetes, Actinobacteria, Tenericutes, and Verrucomicrobia, and/or
at least one bacterium chosen from bacteria of the order Clostridales, Verrucomicrobiales, Aeromonadales, Alteromonadales, ML615J-28, RF32, YS2, of the family Clostridiaceae, Lachnospiraceae, Erysipelotrichaceae, Ruminococcaceae, Bacteroidaceae, Enterococcaceae, Rikenellaceae, Dehalobacteriaceae, Veillonellaceae, and/or
at least one bacterium chosen from bacteria of the genus Faecalibacterium, Akkermansia, Eubacterium, Turicibacter and Oscillospira such as for example Faecalibacterium prausnitzii, Akkermansia muciniphila, Eubacterium halii, Turicibacter sanguinis, Oscillospiraguilliermondii, and/or
at least one prebiotic such as for example at least one prebiotic chosen from galactooligosaccharides, fructooligosaccharides, inulins, arabinoxylans, beta-glucans, lactoglobulins and/or beta-caseins, and/or
at least one polyphenol such as for example at least one polyphenol chosen from quercetin, kaempferol, resveratrol, flavones (such as luteolin), flavan-3-ols (such as catechins), flavanones (such as naringenin), isoflavones, anthocyanidins, proanthocyanidins, and/or
at least one mineral and/or at least one vitamin and/or at least one nutritional agent, and/or
at least one active pharmaceutical ingredient having an effect in the prevention and/or treatment of pathological muscle loss and/or of a disease associated with muscle loss such as, for example, autophagy inhibitors, in particular in the case of cachexia, nusinersen, flunarizine, riluzole, in particular in the case of amyotrophy, vitamin D, sevelamer, polystyrene sulfonate, calcium, calcimetics (in particular cinacalcet), darbopoietin, erythropoietin, furosemide, hydrochlorothiazide, especially in the case of rhabdomyolysis

The invention is now illustrated by examples of useful bacteria according to the invention, methods of culturing these bacteria, examples of compositions containing them and test results demonstrating the effectiveness of the bacteria of the Christensenellaceae family on pathological muscle loss and diseases associated with pathological muscle loss.

### EXAMPLES

### Example 1: Christensenella minuta

The Christensenella minuta bacteria can be cultured according to the operating protocol described below.

1/ Dissolve a dehydrated RCM (“Reinforced Clostridial Medium”) medium in distilled water
2/ Add 0.5 mL/L of resazurin-Na solution (0.1% w/v)
3/ Bring to a boil and cool to room temperature while injecting a gaseous mixture of 80% N₂ and 20% CO₂
4/ Spread the medium under the same gaseous atmosphere in anoxic Hungate-type tubes or in serum vials, then autoclave
5/ Before use, add 1.0 g of sodium carbonate per liter from a sterile anoxic stock solution prepared with a gaseous mixture of 80% N₂ and 20% CO₂
6/ Check the pH of the medium after autoclaving and adjust the pH between 7.3 and 7.5, using a sterile anoxic stock solution of sodium bicarbonate (5% w/v) prepared in a gaseous atmosphere at 80% N₂ and at 20% CO₂.

### Example 2: Christensenella massiliensis

The Christensenella massiliensis bacteria can be cultured according to the operating protocol described below.

1/ Prepare a carboxymethylcellulose (N₂/CO₂) medium by following the instructions below provided by DSMZ (Deutsche Sammlung von Mikroorganismen und Zell-kulturen), presented in Table 1.

TABLE 1

| | | |
|---|---|---|
| Casitone | 30.0 | g |
| Yeast extract | 5.0 | g |
| K₂HPO₄ | 5.0 | g |
| Na-resazurin solution (0.1% w/v) | 0.5 | mL |
| L-Cysteine-HCl × H₂O | 0.5 | g |
| D-Glucose | 4.0 | g |
| Cellobiose | 1.0 | g |
| Maltose | 1.0 | g |
| Na₂CO₃ | 1.0 | g |
| Meat filtrate (see Table 2) | 1000 | mL |

2/ Dissolve the different constituents listed in the table above, except cysteine, carbohydrates and carbonate.
3/ Boil the medium for 1 min, then let it cool to room temperature under a gaseous atmosphere containing 80% N₂ and 20% CO₂.
4/ Add 0.5 g/L of L-cysteine-HCl×H₂O and pour it under the same gaseous atmosphere into Hungate-type tubes (for strains requiring meat particles, introduce these first into the tube; use 1 part meat particles to 4 or 5 parts liquid).

### 5/ Autoclave at 121° C. for 20 min.

6/ After autoclaving, add glucose, cellobiose, maltose and starch from sterile anoxic stock solutions prepared with 100% N₂ gas and carbonate from a sterile anoxic stock solution prepared under gaseous mixtures at 80% N₂ and 20% CO₂.
7/ Adjust the pH of the medium to 7, if necessary.

The composition of the meat filtrate is shown in Table 2.

TABLE 2

| | | |
|---|---|---|
| Ground meat (no fat) | 500.0 | g |
| 1N NaOH | 25.0 | mL |
| Distilled water | 1000 | mL |

The meat filtrate is prepared as follows.

a/ Use lean beef or horse meat.
b/ Remove fat and connective tissue before chopping.
c/ Mix the meat, water and NaOH, then boil for 15 minutes with stirring.
d/ Allow to cool to room temperature, remove fat from the surface and filter, retaining meat particles and filtrate.
e/ Add water to the filtrate to a final volume of 1000.0 mL.

The bacteria must be grown under anaerobic conditions at 37° C.

### Example 3: Christensenella timonensis

The Christensenella timonensis bacteria can be cultivated according to the same procedure as that described in Example 2 for Christensenella massiliensis.

### Example 4: Useful Composition According to the Invention in Liquid Form

An example of a useful composition according to the invention in liquid form is a composition comprising Christensenella minuta 10⁹ CFU/mL in the RCM anaerobic culture medium described above, modified to contain no product of animal origin and enriched with 5% glycerol.

The composition of Example 4 was obtained from an RCB (“research cell bank”) prepared with Christensenella minuta 10¹⁰ CFU/mL and stored frozen at −20° C. in a bag sealed to oxygen.

The frozen composition must be warmed to room temperature until a liquid form is found before use.

### Example 5: Useful Composition According to the Invention in Solid Form

An example of a useful composition according to the invention in freeze-dried form can be obtained by lyophilization of the composition of Example 4 in the frozen state.

### In Vitro Test Demonstrating the Effect of the Invention on Norleucine Synthesis

The objective of this study is to demonstrate the effect of bacteria of the Christensenellaceae family on norleucine in vitro. The study was carried out on norleucine, responsible for the toxicity causing muscle wasting.

Norleucine is a non-proteinogenic amino acid that replaces methionine during protein synthesis. This replacement is toxic: it has been shown to lead to a decrease in the stability of cytochrome P450 (Cirino, P. C., Tang, Y., Takahashi, K., Tirrell, D. A. & Arnold, F. H. Global incorporation of norleucine in place of methionine in cytochrome P450 BM-3 heme domain increases peroxygenase activity. Biotechnol. Bioeng. (2003). doi:10.1002/bit.10718) and a lack of stimulation of muscle synthesis in pigs (Escobar, J. et al. Leucine and alpha-ketoisocaproic acid, but not norleucine, stimulate skeletal muscle protein synthesis in neonatal pigs. J. Nutr. 140, 1418-24 (2010)).

This non-proteinogenic amino acid can be synthesized by different bacterial pathways, including the branched chain amino acid synthesis pathway from pyruvate and alpha-ketobutyrate, where norleucine is a by-product.

The procedure of the study is described below.

### 1/ Fermentation Protocol Using Human Feces Containing Christensenella Spp.:

The donors must not have taken antibiotics during the six months preceding the experiment and have no history of gastrointestinal disorders. The donors were between 18 and 60 years old.
The collection of fresh samples of their feces is obtained in sterile plastic containers, stored in anaerobic bottles containing a 2.5 L sachet of AnaeroGen™ from Oxoid™ (02<0.1%; CO₂: 7-15%). These samples were brought to the laboratory within two hours of their production.
Feces samples were diluted 1/5 (weight/volume) in phosphate buffered saline (1 M) (PBS), pH 7.4. The suspension was homogenized in a stomacher for 120 seconds.
Basic nutrient medium: the basic nutrient medium was prepared from 2 g/L tryptone soy broth, 2 g/L yeast extract, 0.1 g/L NaCl, 0.04 g/L K2 HPO4, 0.01 g/L MgSO3.7 H₂O, 0.01 g/L CaCl₂.6H₂O, 2 g/L NaHCO₃, 0.5 g/L L-cystine HCl, 2 mL/L tween 80, μL/L vitamin K1, 0.05 g/L heme, 0.05 g/L bile salts, 4 ml/L resazarin (pH 7)
Fermentation in a biofermenter: The 20 mL capacity biofermenters contained 18 mL of autoclaved base nutrient medium (121° C. for 15 minutes) poured aseptically into the sterile biofermenters. This system was allowed to stand overnight with oxygen-free nitrogen bubbling through the medium at a rate of 2 mL/min. The pH was maintained between 6.7 and 6.9 using HCl or NaOH (0.5 M). The temperature of each biofermenter was controlled at 37° C. and the contents of the container were homogenized with a magnetic mixer
a mixture of predigested proteins (0.35 g) was added to the containers before inoculation with 2 mL of fecal inocula at TO. The predigested proteins were obtained according to the gastrointestinal digestion protocol adapted from that of Versantvoort et al (2005).
the samples were collected before fermentation (TO) and after 48 hours of fermentation (T48), and frozen at −80° C. until analysis.

### 2/ Quantification of Norleucine

50 μL of samples collected and stored at −80° C. was mixed with 20 μL of Milli-Q water containing internal standards.
The mixture was mixed and filtered through a 5-kDa threshold filter to remove macromolecules.
The metabolites were detected by capillary electrophoresis and time-of-flight mass spectrometry (CE-TOFMS) analyses. The peak detection limit was determined based on the signal-to-noise ratio, S/N=3.

Relative peak area=(metabolite peak area)/(internal standard peak area×amount of sample).

### 3/ Quantification of Christensenella spp.

The DNA contained in the samples was extracted using the NucleoSpin®96 Soil kit from Macherey-Nagel according to the manufacturer's instructions.
The total extracted DNA was then randomly fragmented into 350 bp fragments and then used to build a library using the NEBNext Ultra II kit by New England Biolabs according to the manufacturer's instructions.
The library was then sequenced using 2×150 bp paired-end sequencing on an Illumina HiSeq platform.
The abundance of bacteria was measured by creating a metagenomic species catalog (MGS) from a reference catalog containing 22 M genes. These MGSs were then associated with an appropriate taxonomic level. In the case of Christensenella, these were detected at the genus level and are therefore referred to in this experiment as Christensenella spp.

The relative amount of norleucine and the relative abundance of Christensenella spp were analyzed and correlated, obtaining a linear regression of R=−0.45 (n=18).

The results are shown in Table 3.

TABLE 3

| | Relative abundance of | Relative amount of |
|---|---|---|
| Samples | Christensenella spp (×10⁻²) | Norleucine (×10⁻³) |
| V1 | 7.55 | 0 |
| V2 | 3.18 | 0 |
| V3 | 8.19 | 0 |
| V4 | 2.63 | 19.98 |
| V5 | 1.26 | 5.75 |
| V6 | 2.87 | 23.23 |
| V7 | 7.20 | 5.27 |
| V8 | 2.91 | 0 |
| V9 | 6.32 | 0 |
| V10 | 1.21 | 13.09 |
| V11 | 4.23 | 7.71 |
| V12 | 1.49 | 21.90 |
| V13 | 9.83 | 0 |
| V14 | 4.12 | 1.47 |
| V15 | 6.45 | 0 |
| V16 | 2.02 | 17.22 |
| V17 | 5.23 | 8.66 |
| V18 | 7.57 | 22.62 |

There is a negative correlation between bacteria of the Christensenellaceae family and norleucine, which demonstrates a protective effect of the bacteria of the Christensenellaceae family against the toxicity of norleucine in the enzymatic reactions catalyzed by cytochrome P450 and muscle synthesis inhibition.

Thus, the bacteria of the Christensenellaceae family are capable of acting by reducing the production of markers of pathological muscle loss or of a disease characterized by pathological muscle loss, in particular of norleucine. They can therefore be used to prevent and/or treat pathological muscle loss or diseases characterized by muscle loss.

1. A bacterium of the Christensenellaceae family for its use in the prevention and/or treatment of pathological muscle loss and/or at least one disease characterized by pathological muscle loss in humans or animals.

2. The bacterium of the Christensenellaceae family for its use according to claim 1, in patients or animals exhibiting a decrease in muscle mass correlated with synthesis of norleucine.

3. The bacterium of the Christensenellaceae family for its use according to claim 1 in the treatment and/or prevention of at least one disease characterized by muscle loss chosen from amyotrophy, sarcopenia, pre-sarcopenia, rhabdomyolysis and cachexia.

4. The bacterium of the Christensenellaceae family for its use according to claim 1, characterized in that said bacterium is a bacterium of the genus Christensenella.

5. The bacterium of the Christensenellaceae family for its use according to claim 1, characterized in that said bacterium is selected from Christensenella massiliensis, Christensenella timonensis and Christensenella minuta.

6. A composition comprising at least one bacterium of the Christensenellaceae family for its use in the prevention and/or treatment of pathological muscle loss and/or at least one disease characterized by pathological muscle loss in humans or animals.

7. The composition for its use according to claim 6 in the prevention and/or treatment of at least one disease characterized by muscle loss chosen from amyotrophy, sarcopenia, pre-sarcopenia, rhabdomyolysis and cachexia.

8. The composition for its use according to claim 6, characterized in that it is in liquid form.

9. The composition for its use according to claim 6, characterized in that it is in solid form.

10. The composition for its use according to claim 9, characterized in that the bacteria are present in freeze-dried form.

11. The composition for its use according to claim 6, characterized in that the bacteria present are at least 50% living bacteria (by number).

12. The composition for its use according to claim 6, characterized in that the bacteria present are at least 90% living bacteria (by number).

13. The composition for its use according to claim 6, orally, rectally or inhaled.

14. The composition for its use according to claim 6, characterized in that it is in the form of powder, microencapsulated powder, gelcap, capsule, tablet, lozenge, granules, emulsion, suspension or suppository.

15. The composition for its use according to claim 6, characterized in that it is in a gastro-resistant form.

16. The composition for its use according to claim 6, characterized in that it comprises at least one probiotic and/or at least one prebiotic.

17. The composition for its use according to claim 6, characterized in that it also comprises:
at least one bacterium producing lactic acid and/or at least one other organism promoting the anaerobic conditions necessary for the survival of Christensenellaceae, and/or
and/or
at least one bacterium associated with the Christensenellaceae ecosystem,
at least one bacterium chosen from bacteria of the genus Faecalibacterium,
Akkermansia, Eubacterium, Turicibacter and Oscillospira, and/or
at least one polyphenol, and/or
at least one mineral and/or at least one vitamin and/or at least one nutritional agent, and/or
at least one active pharmaceutical ingredient exhibiting an effect of preventing or treating pathological muscle loss and/or a disease characterized by muscle loss.
